(19) United States
(12) Patent Application Publication
(10) Pub. No.: US 2024/0209060 A1
(43) Pub. Date: Jun. 27, 2024

(54) CELL CULTURE PROCESS FOR FUSION PROTEIN COMPOSITION

(71) Applicant: DR. REDDY’S LABORATORIES LIMITED, Hyderabad (IN)

(72) Inventors: Rama Bhupal Reddy KANDULA, Hyderabad (IN); Suman BANDYOPADHYAY, Secunderabad (IN); Vikas CHANDRAWANSHI, Hyderabad (IN); Sampath kumar VEERAMALLY, Hyderabad (IN); Raghavendra POLISHETTY, Raichur District (IN); Megha BS, Hyderabad (IN)

(21) Appl. No.: 18/285,005

(22) PCT Filed: Mar. 31, 2022
(86) PCT No.: PCT/IN2022/050322

§ 371 Date: Sep. 29, 2023

(30) Foreign Application Priority Data
Mar. 31, 2021 (IN) 202141015005

(51) Int. Cl. C07K 14/705

(52) U.S. Cl. CPC C07K 14/70521; C07K 2319/30

(57) ABSTRACT

The present invention discloses a cell culture method for producing a CTLA-4 fusion protein composition comprising a target % of monomer species of the fusion protein, wherein the cell culture method comprises addition of cysteine in the cell culture medium. The invention further discloses a cell culture process to produce CTLA-4 fusion protein composition with target % of monomer species involving a dual temperature shift with addition of cysteine. Also, the present invention provides a CTLA-4 fusion protein composition comprised of target % of monomer species and/or homodimer species of the CTLA-4 fusion protein.

### FIELD OF INVENTION

The present invention relates to cell culture methods. In particular the cell culture process relates to production of Fc-fusion protein compositions comprising a target % of monomer species of the fusion protein.

### BACKGROUND OF THE INVENTION

Therapeutic glycoproteins form, one of the mainstays in the broader class of biotherapeutics that have been approved for treatment of various human disorders. Although these glycoproteins can be produced in non-mammalian expression systems (e.g. bacterial, yeast, plant, insect expression systems), mammalian expression systems are the preferred production platforms. Protein aggregation is one of the major problems in the use of biotherapeutics as these aggregates affect their immunogenic response and pharmacokinetics.

Protein aggregates are formed due to various covalent and non-covalent interactions. Factors contributing to their formation include cell culture parameters as well as cell culture additives. Among the cell culture parameters, temperature, dissolve oxygen and copper addition have been reported to affect aggregation; and among the additives, cysteine is known to have mild reducing characteristics on disulphide bonds inside the monomer, and thus decrease the product titer. Hence, use of cysteine for reducing protein aggregation was not encouraged (Jing et. al. Process Biochemistry 47 (2012) 69-75).

Abatacept is a recombinant, homodimeric, soluble Fc fusion protein consisting of two identical subunits covalently linked by one disulphide bond. It is designed to block a key costimulatory signal (CD80/CD86) required for Y-cell activation. The abatacept monomer subunit consists of soluble extracellular domain of human cytotoxic T lymphocyte associated antigen 4 (CTLA-4) and a modified version of the Fc domain of human IgG1 which prevents complement-dependent cytotoxicity (CDC) or antibody-dependent cellular cytotoxicity (ADCC). An inter-chain disulphide bond between the monomer subunits in the CTLA-4 portion of the molecule creates a CTLA-4-IgG1 homodimer. This covalent homodimer is the intact active species in an abatacept drug preparation. (Fast et. al. Biochemistry. 2009 Dec. 15; 48(49): 11724-11736).

The present invention relates to a cell culture process to produce a CTLA-4 fusion protein composition comprising target % of monomer species of the fusion protein. The cell culture process comprises uses modulation of cell culture parameters and use of specific additives in cell culture medium.

### SUMMARY OF THE INVENTION

The present invention discloses a cell culture method for producing a CTLA-4 fusion protein composition comprising of monomer and homodimer species of the CTLA-4-IgG fusion protein. The invention provides a cell culture process to produce a CTLA-4 fusion protein composition comprising a target % of monomer and/or homodimer species of the fusion protein using particular range of cysteine in the cell culture medium/feed. The invention further discloses a cell culture process, to produce a CTLA-4 fusion protein composition with target % of monomer and/or homodimer species of the fusion protein, involving a dual temperature shift and addition of cysteine.

### DETAILED DESCRIPTION OF THE INVENTION

The term “about” refers to a range of values that are similar to the stated reference value to a range of values that fall within 25, 20, 19, 18, 17, 16, 15, 14, 13, 12, 11, 10, 9, 8, 7, 6, 5, 4, 3, 2, 1 percent or less of the stated reference value.

The term “addition” or “supplementing” as used herein refers to any supplementation made to cell culture medium/feed to achieve the goals described in this disclosure.

The terms “additives” or “supplements” as used herein refer to any substance added to cell culture medium/feed to achieve the goals described in this disclosure. An “additive” or “supplement” can include sugars (glucose, galactose, or fucose), amino acids (cysteine, serine, or asparagine) etc. The sulphur based additives such as cysteine affects the performance and stability of the cell culture medium.

The terms “cell culture medium”, “culture medium”, “media”, “medium”, as used herein refer to a solution containing nutrients which are required to support the growth of the cells in cell culture. The term would include basal medium which is typically used to support the cell growth during the initial growth phase of cell culture.

The term “cell culture process” as used herein refers to a process of culturing a population of cells that are capable of producing recombinant protein of interest.

The term “dual temperature shift” refers to the change in culture temperature which is performed twice during the cell culture process.

The term “fusion protein composition” or “Fc fusion protein composition” or “CTLA-4 fusion protein composition” refers to a population of fusion protein molecules or fragments thereof that is produced by mammalian cell culture. The population of fusion protein molecules may have one or several post translational modifications (PTM), monomer or dimer.

The term “monomer” refers to the subunits of dimeric or multimeric proteins. Preferably, in the present invention, it refers to the glycosylated polypeptide chain of 357 amino acids with extracellular domain of human Cytotoxic T-Lymphocyte Antigen-4 (CTLA-4) and part of a human immunoglobulin G constant region (C(1), containing the hinge, CH2 and CH3 domains. The term “dimer” refers to the covalently linked through inter-chain disulphide bond.

The term “target % of monomer species” as used herein refers to predetermined characteristic of the fusion protein in terms of the amount of the monomer species in the composition. The target characteristics can be based on existing monographs for that protein, approved specification for the protein by regulatory agencies, or a quality control criterion developed for pharmaceutical preparation of that protein. Same meaning should be ascribed to the phrase “target % of homodimer species”. Also the phrase “target %” is used interchangeably with “predetermines levels”.

The term “temperature shift” refers to the change in culture temperature during the cell culture process.

The present invention relates to CTLA-4-IgG fusion protein composition comprising CTLA-4-IgG fusion protein monomer and CTLA-4-IgG1 fusion protein homodimer. In an embodiment, the present invention provides for a CTLA-4-IgG fusion protein composition comprised of predetermined levels of monomer and/or homodimer species of the CTLA-4-IgG fusion protein.

In another embodiment, the present invention discloses a cell culture method to obtain a CTLA-4-IgG fusion protein composition comprising predetermined levels of monomer and/or homodimer species of the CTLA-4-IgG fusion protein.

Any mammalian cell or cell type which is suitable for expression of recombinant proteins in a cell culture medium may be used for the present invention. Non-limiting examples of mammalian cells that may be used with the present invention include Chinese hamster ovary (CHO) cells, baby hamster kidney (BHK21) cells and murine myeloma cells (NSO and Sp2/0) human retinoblasts (PER.C6 cell line), human embryonic kidney cell line (HEK-293 cell line) (Dumont, J., et al., Human cell lines for biopharmaceutical manufacturing: history, status, and future perspectives. Crit Rev Biotechnol, 2016. 36(6): p. 1110-1122). In a preferred embodiment, CHO cell lines expressing recombinant proteins may be used in accordance with the present invention.

Cell culture medium is understood by those skilled in the art to refer to a nutrient solution in which cells, such as animal or mammalian cells, are grown. A cell culture medium generally includes one or more of the following components: an energy source (e.g., a carbohydrate such as glucose); amino acids; vitamins; lipids or free fatty acids; and trace elements, e.g., inorganic compounds or naturally occurring elements in the micromolar range. Cell culture medium can also contain additional components, such as hormones and other growth factors (e.g., insulin, transferrin, epidermal growth factor, serum, and the like); salts (e.g., calcium, magnesium and phosphate); sugars (e.g. mannose, galactose, fucose); amino acids (glutamine); buffers (e.g., HEPES); nucleosides and bases (e.g., adenosine, thymidine, hypoxanthine); antibiotics (e.g., gentamycin); and cell protective agents (e.g., a Pluronic polyol (Pluronic F68). Commercially available media can be utilized in accordance with the present invention, for example, Dulbecco's Modified Eagles Medium (DMEM, Sigma-Aldrich); RPMI-1640 Medium (Sigma-Aldrich); EX-CELL® Advanced CHO Fed-batch Medium (Sigma-Aldrich); Cell Boost™ M 7a and 7b (GE Healthcare Bio-Sciences AB). One skilled in the art would appreciate that some cell culture media are suited to support cells through their initial growth phase (basal medium) while some sustain cells through the later growth phase and production phase of cell culture (feed medium), and would be able to choose appropriate culture medium.

In an embodiment the cell culture method of the present invention includes addition of specific cell culture medium additives to obtain CTLA-4-IgG fusion protein composition comprising predetermined levels of monomer and/or homodimer species of the CTLA-4-IgG fusion protein.

In an embodiment, the cell culture process of the present invention would comprise addition of sulphur based additives. In particular, the present invention encompasses use of cysteine in the cell culture medium to obtain the claimed CTLA-4-IgG fusion protein composition. In a preferred embodiment, the present invention encompasses use of particular levels of cysteine in the cell culture medium to obtain CTLA-4-IgG fusion protein composition comprising predetermined levels of monomer and/or homodimer species of the CTLA-4-IgG fusion protein.

In an embodiment, the cell culture process of the present invention would comprise more than one temperature shift, wherein the individual temperature shift might be result in subsequent lower temperature or higher temperature.

In an embodiment, the present invention provides a cell culture process to produce CTLA-4 fusion protein composition comprising of monomer and homodimer species of the protein, the process comprising,
supplementation of sulphur based additives,
subjecting the cell culture to a dual temperature shift
   thereby obtaining a composition of the fusion protein comprising of target % of monomer species.

In an embodiment, the cell culture is subjected to a dual temperature shift, the first temperature shift is performed by lowering the temperature by about 3° C. and the second temperature shift is performed by lowering the temperature by about 4° C.

In an embodiment, the present invention provides a cell culture process to produce CTLA-4 fusion protein composition comprising of monomer and homodimer species of the protein, the process comprising,
addition of cysteine to the cell culture,
subjecting the cell culture to a first temperature for a first period of time,
lowering the temperature to a second temperature and culturing for a second period of time,
further lowering the temperature to a third temperature and culturing for a third period of time,
recovering the fusion protein from the culture.
   wherein the concentration of cysteine added does not exceed about 7 mM, thereby obtaining a CTLA-4 fusion protein composition comprising a target % of monomer species.

In an embodiment, the present invention provides a cell culture process to produce a composition of CTLA-4 fusion protein comprising of monomer and homodimer species of the protein, the process comprising,
addition of cysteine to the cell culture,
subjecting the cell culture to a dual temperature shift,
wherein the first temperature shift is from 37° ° C.to 34° C. on day 5 and the second temperature shift is from 34° C. to 30° C. on day 9
   wherein the concentration of cysteine added does not exceed 7 mM
   thereby obtaining CTLA-4 fusion protein composition comprising of about 10% monomer species.

In an embodiment, the present invention further provides a cell culture process to produce a CTLA-4 fusion protein composition comprising of monomer and homodimer species of the protein, the process comprising
a dual temperature shift,
supplementing the medium with glucose,
addition of cysteine to the cell culture,
recovering the fusion protein from the culture,
   wherein the concentration of cysteine added does not exceed 7 mM
   thereby obtained a CTLA-4 fusion protein composition comprising a target of about 10% monomer species.

In an embodiment, the present invention further provides a cell culture process to produce a CTLA-4 fusion protein composition, the process comprising
addition of cysteine to the cell culture,
supplementing the medium with glucose,
subjecting the cell culture to a dual temperature shift,
the first temperature shift is from 37° C. to 34° C. on day 5,
the second temperature shift is from 34° C. to 30° C. on day 9
   wherein the concentration of cysteine added does not exceed 7 mM
   thereby obtained a CTLA-4 fusion protein composition comprising a target of about 10% monomer species.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLE

The CTLA-4-IgG fusion protein producing CHO cells were cultured with seeding cell density of 0.5 million cells/ml in cell culture medium. The cell culture medium was supplemented with glucose, which was made up to a concentration of 5 g/L every day. To study the effect of different concentrations of cysteine, feed mediums having cysteine concentration ranging from 0 to 27.52 mM were used. The cell culture was initiated at temperature 37° C. and a dual temperature shifts were performed. The first shift from 37° ° C. to 34° C. was done on day 5 and second shift from 34° C. to 30° C. was performed on day 9 of the cell culture. The cell cultures were harvested on day 13. The % of monomer species in the fusion protein compositions produced under various conditions is given in Table 1.

TABLE 1
Effect of Cysteine supplementation on monomer
species in CTLA-4-IgG fusion protein.

| Feed | Cysteine | | Replicates | % Monomer |
|---|---|---|---|---|
| Medium | Concentration | | (N) | species |
| 1 | 0 | mM | 4 | 10.9 ± 1.4 |
| 2 | 1.72 | mM | 4 | 10.4 ± 1.8 |
| 3 | 3.44 | mM | 4 | 10.6 ± 2.7 |
| 4 | 6.88 | mM | 4 | 9.6 ± 1.3 |
| 5 | 13.76 | mM | 4 | 14.8 ± 3.3 |
| 6 | 27.52 | mM | 2 | 25.7 ± 0.1 |

1. A cell culture process to produce a CTLA-4 fusion protein composition comprising of monomer and homodimer species of the protein, the process comprising,
supplementation of sulphur based additives,
subjecting the cell culture to a first temperature for a first period of time,
lowering the temperature to a second temperature and culturing at the second temperature for a second period of time,
further lowering the temperature to a third temperature and culturing at the third temperature for a third period of time,
recovering the fusion protein from the culture
thereby obtaining a composition of the fusion protein comprising of target % of monomer species.

2. The cell culture process of claim 1, wherein the sulphur based additives is cysteine.

3. A cell culture process to produce CTLA-4 fusion protein composition comprising of monomer and homodimer species of the protein, the process comprising,
supplementing cell culture medium with cysteine not exceeding 7 mM,
subjecting the cell culture to a first temperature for a first period of time,
lowering the temperature to a second temperature and culturing at the second temperature for a second period of time,
further lowering the temperature to a third temperature and culturing at the third temperature for a third period of time,
recovering the fusion protein from the culture
thereby obtaining a CTLA-4 fusion protein composition comprising a target % of monomer species.

4. The cell culture process according to claim 1, wherein the second temperature is lower than the first temperature by about 3° C. and the third temperature is lower than the second temperature by about 4° C.

5. The cell culture process according to claim 1, wherein the first temperature of about 37° C. is lowered to second temperature of about 34° C. on day 5, and the second temperature is lowered to third temperature of about 30° ° C.on day 9.

6. The cell culture process as claimed in claim 1, which further comprises of supplementing the medium with glucose.

7. The cell culture process as claimed in claim 1, wherein the target % of monomer species is not exceeding about 10%.

8. The cell culture process of claim 1, wherein the CTLA-4 fusion protein is abatacept.

9. The cell culture process according to claim 3, wherein the second temperature is lower than the first temperature by about 3° C. and the third temperature is lower than the second temperature by about 4ºC.

10. The cell culture process according to claim 3, wherein the first temperature of about 37° C. is lowered to second temperature of about 34° C. on day 5, and the second temperature is lowered to third temperature of about 30° C. on day 9.

11. The cell culture process as claimed in claim 3, which further comprises of supplementing the medium with glucose.

12. The cell culture process as claimed in claim 3, wherein the target % of monomer species is not exceeding about 10%.

13. The cell culture process of claim 3, wherein the CTLA-4 fusion protein is abatacept.
